(19) United States
(12) Patent Application Publication
(10) Pub. No.: US 2007/0122384 A1
(43) Pub. Date: May 31, 2007

(54) Method for treating inflammatory bowel disease

(75) Inventor: Jan K. Ohrstrom, Mercer Island, WA (US)

Correspondence Address: NOVO NORDISK, INC.;PATENT DEPARTMENT, 100 COLLEGE ROAD WEST, PRINCETON, NJ, 08540, US

(73) Assignee: ZYMOGENETICS, INC., Seattle, WA (US)

(21) Appl. No.: 11/641,163

(22) Filed: Dec. 19, 2006

Related U.S. Application Data
(63) Continuation of application No. 10/525,271, filed on Sep. 15, 2005, now abandoned.
(60) Provisional application No. 60/405,388, filed on Aug. 23, 2002.

(51) Int. Cl. A61K 38/36; A61K 38/21

(52) U.S. Cl. 424/85.5; 514/12

(57) ABSTRACT

A method for treating inflammatory bowel disease by administering interferon beta in conjunction with factor XIII to a mammal afflicted with the disease. The IBD may either be ulcerative colitis or Crohn's disease. The factor XIII used may be recombinant or nonrecombinant. The interferon beta used can be naturally occurring interferon beta, interferon beta-1a or interferon beta-1b.

### CROSS-REFERENCE TO RELATED PATENT APPLICATIONS

This patent application is a continuation of copending U.S. patent application Ser. No. 10/525,271, filed Feb. 22, 2005 which is a National Phase 371 filing of PCT/US2003/026591 filed Aug. 25, 2003 and claims the benefit of U.S. Provisional Patent Application No. 60/405,388, filed Aug. 23, 2002.

### BACKGROUND OF THE INVENTION

The teachings of all of the references cited herein are incorporated in their entirety herein by reference.

Inflammatory bowel diseases (IBD) are defined by chronic, relapsing intestinal inflammation of obscure origin. IBD refers to two distinct disorders, Crohn's disease and ulcerative colitis (IC). Both diseases appear to result from the unrestrained activation of an inflammatory response in the intestine. This inflammatory cascade is thought to be perpetuated through the actions of proinflammatory cytokines and selective activation of lymphocyte subsets. In patients with IBD, ulcers and inflammation of the inner lining of the intestines lead to symptoms of abdominal pain, diarrhea, and rectal bleeding. Ulcerative colitis occurs in the large intestine, while in Crohn's, the disease can involve the entire gastrointestinal (GI) tract as well as the small and large intestines. For most patients, IBD is a chronic condition with symptoms lasting for months to years. It is most common in young adults, but can occur at any age. It is found worldwide, but is most common in industrialized countries.

The clinical symptoms of IBD are intermittent rectal bleeding, crampy abdominal pain, weight loss and diarrhea. Diagnosis of IBD is based on the clinical symptoms and the use of a barium enema, but direct visualization (sigmoidoscopy or colonoscopy) is the most accurate test. Protracted IBD is a risk factor for colon cancer, and treatment of IBD can involve medications and surgery.

Some patients with UC only have disease in the rectum (proctitis). Others with UC have disease limited to the rectum and the adjacent left colon (proctosigmoiditis). Yet others have UC of the entire colon. Symptoms of UC are generally more severe with more extensive disease (larger portion of the colon involved with disease). The prognosis for patients with disease limited to the rectum (proctitis) or UC limited to the end of the left colon (proctosigrnoiditis) is better then that of full colon UC. Brief periodic treatments using oral medications or enemas may be sufficient. In those with more extensive disease, blood loss from the inflamed intestines can lead to anemia, and may require treatment with iron supplements or even blood transfusions. Rarely, the colon can acutely dilate to a large size when the inflammation becomes very severe. This condition is called toxic megacolon. Patients with toxic megacolon are extremely ill with fever, abdominal pain and distention, dehydration, and malnutrition. Unless the patient improves rapidly with medication, surgery is usually necessary to prevent colon rupture.

Crohn's disease can occur in all regions of the gastrointestinal tract. With this disease intestinal obstruction due to inflammation and fibrosis occurs in a large number of patients. Granulomas and fistula formation are frequent complications of Crohn's disease. Disease progression consequences include intravenous feeding, surgery and colostomy. Colon cancer is a known complication of chronic IBD. It is increasingly common in those patients who have had extensive IBD over many years. The risk for cancer begins to rise significantly after eight to ten years of IBD.

IBD may be treated medicinally. The most commonly used medications to treat IBD are anti-inflammatory drugs such as the salicylates. The salicylate preparations have been effective in treating mild to moderate disease. They can also decrease the frequency of disease flares when the medications are taken on a prolonged basis. Examples of salicylates include sulfasalazine, olsalazine, mesalamine and azulfidine. All of these medications are given orally in high doses for maximal therapeutic benefit. These medicines are not without side effects. Azulfidine can cause upset stomach when taken in high doses, and rare cases of mild kidney inflammation have been reported with some salicylate preparations. Corticosteroids are more potent and faster-acting than salicylates in the treatment of IBD, but potentially serious side effects limit the use of corticosteroids to patients with more severe disease. Side effects of corticosteroids usually occur with long term use. They include thinning of the bone and skin, infections, diabetes, muscle wasting, rounding of faces, psychiatric disturbances, and, on rare occasions, destruction of hip joints.

In IBD patients that do not respond to salicylates or corticosteroids, medications that suppress the immune system are used. Examples of immunosuppressants include azathioprine and 6-mercaptopurine. Immunosuppressants used in this situation help to control IBD and allow gradual reduction or elimination of corticosteroids. However, immunosuppressants render the patient immuno-compromised and susceptible to many other diseases.

It is apparent that the current therapies to treat IBD have been inadequate. Thus, there is a need for novel, more effective therapies to treat IBD.

### DESCRIPTION OF THE INVENTION

The present invention fills this need by providing for a method for treating inflammatory bowel disease comprising administering to a patient with inflammatory bowel disease a therapeutically effective amount of interferon beta in conjunction with factor XIII.

Definitions

The phrase “symptoms of IBD” is herein defined as detected symptoms such as abdominal pain, diarrhea, rectal bleeding, weight loss, fever, loss of appetite, and other more serious complications, such as dehydration, anemia and malnutrition. A number of such symptoms are subject to quantitative analysis (e.g., weight loss, fever, anemia, etc.). Some symptoms are readily determined from a blood test (e.g. anemia) or a test that detects the presence of blood (e.g., rectal bleeding). The phrase “wherein said symptoms are reduced” refers to a qualitative or quantitative reduction in detectable symptoms, including but not limited to a detectable impact on the rate of recovery from disease (e.g., rate of weight gain).

The phrase “at risk for IBD” is herein defined as encompassing the segment of the world population that has an increased risk for IBD. IBD is most commonly found in young adults, but can occur at any age. It occurs worldwide, but is most common in the United States, England, and northern Europe. It is especially common in people of Jewish descent. An increased frequency of this condition has been recently observed in developing nations.

The phrase “administered to or at the lumen” is herein defined as delivery to the space in the interior of the intestines. Such delivery can be achieved by a variety of routes (e.g., oral, rectal, etc.) in a variety of vehicles (e.g., tablet, suppository, etc.).

Factor XIII, also known as fibrin-stabilizing factor, circulates in the plasma at a concentration of about 20 mg/ml. The protein exists in plasma as a tetramer comprised of two A subunits and two B subunits. Each subunit has a molecular weight of 83,000 Da, and the complete protein has a molecular weight of approximately 330,000 Da. Factor XIII catalyzes the cross-linkage between the γ-glutamyl and ε-lysyl groups of different fibrin strands. The catalytic activity of factor XIII resides in the A subunits. The B subunits act as carriers for the A subunits in plasma factor XIII. Recombinant factor XIII can be produced according to the process described in European Patent No. 0 268 772 B1. The level of factor XIII in the plasma can also be increased by administering a factor XIII concentrate derived from human placenta, or derived from human plasma called FIBROGAMMIN® (Aventis Corp.) or by administration of recombinant factor XIII.

A pharmaceutical composition comprising factor XIII can be formulated according to known methods to prepare pharmaceutically useful compositions, whereby the therapeutic proteins are combined in a mixture with a pharmaceutically acceptable carrier. A composition is said to be a “pharmaceutically acceptable carrier” if its administration can be tolerated by a recipient patient. A suitable pharmaceutical composition of factor XIII will contain 1 mM EDTA, 10 mM Glycine, 2% sucrose in water. An alternative formulation will be a factor XIII composition containing 20 mM histidine, 3% wt/volume sucrose, 2 mM glycine and 0.01% wt/vol. polysorbate, pH 8. The concentration of factor XIII should preferably be 1-10 mg/mL, more preferably about 5 mg/mL.

Other suitable carriers are well known to those in the art. See, for example, Gennaro (ed.), Remington's Pharmaceutical Sciences, 19th Edition (Mack Publishing Company 1995).

Factor XIII can be administered intravenously, intramuscularly or subcutaneously, rectally by enema or by lavage to treat inflammatory bowel disease. The levels of factor XIII in an individual can be determined by assays well known in the art such as the BERICHROM® F XIII assay (Dade Behring Marburg GmbH, Marburg, Germany). The normal adult has an average of about 45 ml of plasma per kg of body weight. Each liter of blood has 1000 units (U) of factor XIII. A dose of 0.45 U/kg would raise the level of factor XIII by about 1% compared to normal. One unit of factor XIII is about 10 micrograms (mcg) of recombinant factor XIII, which contains only the dimerized, ‘A’ subunit. Thus, to raise the level of factor XIII by 1%, one would administer about 4.5 mcg of the A2 subunit per kilogram weight of the individual. So to raise the level 30% of normal, one would administer 13.5 U/kg. For a 75 kg individual this would be about 1,012.5 U. Some patients may have consumptive coagulopathies that involve factor XIII losses. In such cases, a higher dosing (e.g., 1-2 U/kg-%) or multiple dosing of factor XIII (e.g., 1-2 U/kg-%-day) may be required.

According to the present invention, factor XIII is administered in conjunction with interferon beta to treat inflammatory bowel disease. Interferon beta is currently being marketed in two forms. AVONEX®, interferon beta-1a, is produced by Biogen, Inc., Cambridge, Mass. and BETASERON, interferon-1b is produced by Berlex Laboratories, Richmond, Calif.

Interferon beta-1a, AVONEX®, can be administered to doses ranging 30 mcg to 75 mcg per patient per week. However, optimal dosage may vary for each individual. Factor XIII can be administered at the same time, before or after administration of interferon beta. Interferon beta-1a is produced by recombinant DNA technology. It is a 166 amino acid glycoprotein with a predicted molecular weight of approximately 22.5 kD. It is produced by Chinese Hamster Ovary cells into which the human interferon beta gene has been introduced.

Using the World Health Organization (WHO) natural interferon beta standard, Second International Standard for Interferon, Human Fibroblast (Gb-23-902-5341), interferon beta-1a (AVONEX®), has a specific activity of approximately 200 million international units (IU) of antiviral activity per mg.

The other form of interferon beta currently being marketed is interferon beta-1b, BETASERON®. Interferon beta-1b is a purified, sterile, lyophilized protein product also produced by recombinant DNA technology. Interferon beta-1b is manufactured by bacterial fermentation of a strain of Escherichia coli that bears a genetically engineered plasmid containing the gene for human interferon beta ser17. The native gene was obtained from human fibroblasts and altered in a way that substitutes serine for the cysteine residue found at position 17. Interferon beta-1b has 165 amino acids and an approximate molecular weight of 18.5 kD.

The specific activity of interferon beta-1b is 32 million IU/mg. The recommended dosage of interferon beta-1b is 0.05 to 0.25 mg every other day. The preferred dose is 0.25 mg administered every other day or more until symptoms improve. The dosages of interferon beta may be increased until unpleasant side effects such as flu-like symptoms develop.

In carrying out the methods of the present invention, the factor XIII and interferon beta may be administered to various mammalian species, such as monkeys, cats, dogs, rats, humans, etc., in need of such treatment. The exact rout of administration, dosage form, amount to be administered and method of administration may be readily determined by one of ordinary skill in the art.

Factor XIII and interferon beta can also be administered in conjunction with other drug therapies that have been used in the past to treat inflammatory bowel disease including salicylates such as sulfasalazine, olsalazine, mesalamine and azulfidine, corticosteroids, immunosuppressants such as azathioprine and 6-mercaptopurine, and antibodies to tumor necrosis factor or a soluble receptor to tumor necrosis factor.

There are several animal models that can partially mimic chronic ulcerative colitis which can be used to evaluate the methods of the present invention The most widely used model is the 2,4,6-trinitrobenesulfonic acid/ethanol (TNBS) induced colitis model, which induces chronic inflammation and ulceration in the colon. When TNBS is introduced into the colon of susceptible mice via intra-rectal instillation, it induces a T-cell mediated immune response in the colonic mucosa, in this case leading to a massive mucosal inflammation characterized by the dense infiltration of T-cells and macrophages throughout the entire wall of the large bowel. Moreover, this histopathologic picture is accompanied by the clinical picture of progressive weight loss (wasting), bloody diarrhea, rectal prolapse, and large bowel wall thickening (Neurath et al., Intern. Rev. Immunol. 19:51-62, 2000).

Another colitis model uses dextran sulfate sodium (DSS), which induces an acute colitis manifested by bloody diarrhea, weight loss, shortening of the colon and mucosal ulceration with neutrophil infiltration. DSS-induced colitis is characterized histologically by infiltration of inflammatory cells into the lamina propria, with lymphoid hyperplasia, focal crypt damage, and epithelial ulceration. These changes are thought to develop due to a toxic effect of DSS on the epithelium and by phagocytosis of lamina propria cells and production of TNF-alpha and IFN-gamma. DSS is regarded as a T cell-independent model because it is observed in T cell-deficient animals such as SCID mice.

What is claimed is:

1. A method for treating inflammatory bowel disease (IBD) in a mammal comprising administering a therapeutically effective amount of interferon beta in conjunction with factor XIII.

2. The method of claim 1 wherein the factor XIII is recombinant factor XIII.

3. The method of claim 1 wherein the IBD is ulcerative colitis.

4. The method of claim 1 wherein the IBD is Crohn's disease.

5. The method of claim 1 wherein the mammal is further treated with a drug selected from the group consisting of salicylates including sulfasalazine, olsalazine, mesalamine and azulfidine, corticosteroids, immunosuppressants such as azathioprine and 6-mercaptopurine, and antibodies to tumor necrosis factor.

6. The method of claim 1 wherein the interferon beta is interferon beta-1a.

7. The method of claim 1 wherein the interferon beta is interferon beta-1b.

8. The method of claim 1 wherein the mammal is a human.

9. The method of claim 1 wherein the factor XIII is administered at the same time as the interferon beta.

10. The method of claim 1 wherein the factor XIII is administered before the interferon beta.

11. The method of claim 1 wherein the factor XIII is administered after the interferon beta.

12. The method of claim 1 wherein the factor XIII is administered intravenously.

13. The method of claim 1 wherein the factor XIII is administered to or at the lumen.
